(19) United States
(12) Patent Application Publication
(10) Pub. No.: US 2025/0064356 A1
(43) Pub. Date: Feb. 27, 2025

(54) MEDICAL SENSOR

(71) Applicant: Covidien LP, Mansfield, MA (US)

(72) Inventors: Sarah L. Hayman, Boulder, CO (US); Bonaire G. Berry, Denver, CO (US); Abel Valdes, Lafayette, CO (US); Winborne L. Hamlin, Westminster, CO (US); Shao-Nung Liang, Broomfield, CO (US)

(21) Appl. No.: 18/811,457

(22) Filed: Aug. 21, 2024

Related U.S. Application Data
(60) Provisional application No. 63/520,846, filed on Aug. 21, 2023.

(51) Int. Cl. A61B 5/1455; A61B 5/00

(52) U.S. Cl. CPC A61B 5/14552; A61B 5/6832; A61B 2560/0412; A61B 2562/0238; A61B 2562/0242; A61B 2562/12

(57) ABSTRACT

An oximetry sensor includes a light source configured to emit first light into a tissue of a patient, the first light comprising a first light wavelength greater than a threshold wavelength and a light filter. The light filter is configured to receive the first light from the tissue of the patient, filter a second light wavelength from at least one of the received first light or ambient light. The second light wavelength is less than or equal to the threshold wavelength. The light filter is also configured to output a filtered light that includes the received first light from the tissue. The oximetry sensor also includes a detector configured to detect the filtered light, the filtered light being indicative of an oxygen saturation level of the patient.

### CROSS-REFERENCE TO RELATED APPLICATIONS

This application is related to U.S. Provisional Application No. 63/520,846, filed Aug. 21, 2023, entitled “MEDICAL SENSOR,” which is hereby incorporated by reference in its entirety for all purposes.

### TECHNICAL FIELD

The present disclosure generally relates to medical sensors.

### BACKGROUND

Medical sensors are used to monitor one or more physiological parameters of a patient. For example, an oxygen saturation monitoring system with an oxygen saturation sensor or oximetry sensor, such as a pulse oximetry sensor or a regional oximetry sensor, is configured to monitor oxygen saturation levels of a patient. In some examples, a noninvasive pulse oximetry sensor is placed on a patient to measure the oxygen saturation level of the patient via photoplethysmography. When the oxygen saturation level of the patient decreases to reach a desaturation threshold, the oxygen saturation monitoring system may output an indication that the patient is experiencing oxygen desaturation.

### SUMMARY

The present disclosure describes example devices and techniques and devices for monitoring a physiological parameter of a patient in an environment including ambient light.

In one example, an oximetry sensor includes: a light source configured to emit first light into a tissue of a patient, the first light comprising a first light wavelength greater than a threshold wavelength; a light filter configured to: receive the first light from the tissue of the patient; filter a second light wavelength from at least one of the received first light or ambient light, wherein the second light wavelength is less than or equal to the threshold wavelength; and output a filtered light that includes the received first light from the tissue; and a detector configured to detect the filtered light, the filtered light being indicative of an oxygen saturation level of the patient.

In another example, a method of manufacturing an oximetry sensor includes: attaching a light source to a sensor patch of the oximetry sensor, wherein the light source is configured to emit first light into a tissue of a patient, the first light comprising a first light wavelength greater than a threshold wavelength, wherein the sensor patch is configured to be applied to a region of skin of a patient; disposing a light filter to cover over at least a portion of an active area of a detector, wherein the light filter is configured to: receive the first light from the tissue of the patient; filter a second light wavelength from at least one of the received first light or ambient light, wherein the second light wavelength is less than or equal to the threshold wavelength; and output a filtered light that includes the received first light from the tissue; and attaching the detector to the sensor patch, wherein the detector is configured to detect the filtered light, the filtered light being indicative of an oxygen saturation level of the patient.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 a perspective view of an example system configured to monitor one or more physiological parameters of a patient, including an example pulse oximetry sensor patch.

FIG. 2 is a perspective view of an example patient monitoring sensor.

FIG. 3 is a top view of another example patient monitoring sensor.

FIG. 4 is an exploded top perspective view of another example patient monitoring sensor.

FIG. 5A is an exploded bottom perspective view of another example patient monitoring sensor.

FIG. 5B is an exploded bottom perspective view of another example patient monitoring sensor.

FIG. 5C is an exploded bottom perspective view of another example patient monitoring sensor.

FIG. 5D is an exploded top perspective view of another example patient monitoring sensor.

FIG. 6 is a perspective view of an example detector package that includes a light filter.

FIG. 7 is a graph including a plurality of plots illustrating spectral output of a plurality of light sources overplotted with a spectral responsivity of an example detector including an example light filter.

FIG. 8 is a flowchart illustrating an example technique of making a patient monitoring sensor.

### DETAILED DESCRIPTION

In general, aspects of the present disclosure relate to devices and techniques for monitoring a physiological parameter, such as, but not limited to, oxygen saturation, of a subject, e.g., a patient.

Optical sensors may be attached to a patient, e.g., on a skin surface, to sense one or more patient physiological parameters. For example, optical sensors may be used to sense temperature, blood pressure, blood flow, heart rate, respiration, or oxygen saturation. Certain examples of the present disclosure describe devices and techniques with reference to pulse oximetry sensors. However, devices and techniques according to the present disclosure may be used with any suitable optical sensor configured to generate a signal indicative of a physiological parameter, such as, but not limited to, a regional oxygen saturation sensor.

Oximeters, such as regional oximeters or pulse oximeters, may be used to non-invasively monitor oxygen saturation by detecting light transmitted through a target region. An oximeter includes an oximetry sensor configured to sense and send signals to a control circuitry. For example, a pulse oximetry sensor or a regional oximetry sensor may include one or more light sources, which may be photoemitters, configured to emit light in a wavelength range (e.g., specified wavelength range), and the pulse oximetry sensor or the regional oximetry sensor may also include one or more detectors (also referred to herein as “a detector” to facilitate discussion) configured to sense the light emitted by the photoemitter after the light is transmitted through tissue of the patient. The oximetry sensor may send signals indicative of the light that is received by the detectors. The control circuitry may receive the signals and determine oxygen saturation and/or other patient physiological parameters based on the signals. The oximetry sensor may be applied in contact with a region of a patient's skin, for example, as a patch that secures or houses one or more photoemitters and one or more detectors. Thus, an oximetry sensor patch may enable detection of oxygen saturation in blood flowing through the region.

Pulse oximetry sensor patches may be applied on or about a finger, or a portion of an extremity of the patient. Some patients may exhibit relatively poor perfusion at extremities, and regional oximetry sensors may be applied at other locations, for example, on a patient's forehead, on a patient's back (e.g., over a kidney of the patient), on a patient's abdomen (e.g., near about the patient's mesentery). A forehead regional oximetry sensor patch may, for example, be applied directly above the left or right eye. Due to the proximity of such a location to the brain, a sensor applied on the forehead may provide relatively faster measurement of oxygen saturation than a sensor applied to a finger (or extremity), and may still measure oxygen saturation when a patient's extremities are poorly perfused. The light path may differ depending on the location of the sensor. For example, transmitted light may be sensed when the sensor is positioned on or about the finger or the portion of the extremity, while reflected light may be sensed when the sensor is positioned on the forehead. In some examples, oximetry sensors may provide critical patient data that clinicians may rely on to make quick response decisions that affect the health and life of a patient.

Oximetry sensors may be used in an environment including ambient light, e.g., light that is not emitted by the photoemitter and/or light source of the sensor. Ambient light is detected by the detector of the oximetry sensor and, when bright enough, can cause erroneous readings and/or signal dropouts (e.g., a lack of signal due to saturation of the front-end electronics or the detected light being outside of the dynamic range of the electronics). For example, at least a portion of ambient light may pass, or transmit, through a sensor patch or bandage to a detector of the sensor. Additionally, ambient light may at least partially transmit through the patient's tissue (e.g., at an area not covered by a patch or bandage) and diffuse (e.g., diffusely transmit, thereby increase a volume or area over which the ambient light propagates) through the tissue to a detector of the sensor. For example, ambient light may transmit into a patient's tissue that is unblocked at an edge of a sensor patch nearest to a detector of the sensor. The amount of ambient light that reaches the detector may substantially follow the Beer-Lambert law, e.g., I=Iₒe^{-(ua-us)t}, where “Io” is the amount of ambient light entering the patient's tissue, “ua” is an optical absorption coefficient of the patient's tissue (e.g., modeling tissue having a homogeneous and isotropic absorption), “us” is an optical scattering coefficient of the patient's tissue (e.g., modeling tissue having a homogeneous and isotropic scattering), and “t” is the path length of travel of the light through the tissue.

In some examples, ambient light may propagate to a detector via any number of single or multiple specular or diffuse reflections and/or single or multiple specular or diffuse transmissions through one or more materials to arrive at a detector, e.g., multiple reflections of ambient light that is “let in” via gaps between the sensor patch or bandage and the skin of the patient, such as with applying a sensor to a finger or digit of the patient where it may be difficult for the patch or bandage to conform to the skin of the finger or digit.

Some environments, such as hospitals, may include multiple sources of ambient light, such as overhead lighting which may include fluorescent, incandescent, or light emitting diode (LED) sources, neonatal warmers, and sunlight from windows. Additionally, some environments may have more or less ambient light. For example, an operating room may have both integrated ceiling lights and bright surgical lights, which typically may be fluorescent lights and LED lights. In some examples, an operating table in an operating room may be illuminated with an illuminance of about 800 lux, or about 3000 lux, or more. In some examples, a patient using a patient monitoring sensor may be a cardiac patient in a cardiac operating room in which skin adjacent to the sensor may be illuminated with about 3000 lux or more of ambient light. In other examples, a patient may be an infant in a neonatal intensive care unit, a child in a pediatric intensive care unit, or a child or young adult in a cardiac intensive care unit including fluorescent or LED ceiling lights, neonatal warmers and isolettes, UV bilirubin lights, UV from sunlight through windows, and procedure lights (e.g., halogen and/or LED lights).

When possible, clinicians may use alternatives or “workarounds” to mitigate the effects of ambient light, especially in an operating room environment where lights are bright. For example, a clinician may cover the sensor on the patient with relatively opaque towels.

There may also be signal processing techniques to subtract and/or filter out the detected ambient light, e.g., by the digital/analog electronics. However, signal processing to filter out detected ambient light may be limited because there may be a maximum amount (or magnitude, or amplitude, or brightness or radiance) of ambient that can be filtered before the sensor signal is overwhelmed.

Additionally, some applications may benefit from an oximetry sensor having a smaller area, or “footprint,” so that the sensor can fit on smaller patients, such as regional oximetry sensor for infants, such as infants born before their due date or a full gestation period, e.g., from about 28 to 32 weeks gestation age. However, reducing the area of the sensor may degrade ambient light performance, e.g., increase negative effects due to ambient light. For example, ambient light may have a shorter path through sensor patch and/or bandage material to a detector of the sensor that has a reduced size or area. In other words, the sensor patch and/or bandage may reduce (e.g., block, filter, absorb) a portion of ambient light. The size and/or opacity of the patch and/or bandage may affect the amount of ambient light incident on an active area of a detector.

In examples described herein, an oximetry sensor includes a light source configured to emit light having a wavelength greater than a threshold wavelength to, or into, tissue of a patient, a detector configured to detect and/or receive the light from tissue of the patient after the light was emitted to, or into, the tissue of the patient from the light source, and a light filter between the light source and the detector. In some examples, the light filter is adjacent to an active area of the detector such that all light received by the active area of the detector passes through, or interacts with, the filter. The light filter is configured to receive the light from the tissue of the patient and ambient light including a wavelength less than or equal to the threshold wavelength, and to pass (or output) filtered light that includes at least a portion of the light from the tissue and filters out at least a portion of the ambient light (e.g., blocks, absorbs, redirects, or otherwise prevents at least a portion of the ambient light from being received by the detector). The light filter may be configured to filter out received ambient light, and pass light received from the tissue of the patient, based on the wavelength of the light. For example, the light filter may be configured to pass (e.g., receive and output to the detector) light having a wavelength that is greater than a threshold wavelength and filter out (receive and reduce or prevent output to the detector) light having a wavelength that is less than or equal to the threshold wavelength. In some examples, the light filter may be configured to filter out light having a wavelength that is less than or equal to the threshold wavelength by reducing the amount of light having a wavelength that is less than or equal to the threshold wavelength, e.g., rather than filtering out completely. For example, the light filter may filter out light having a wavelength that is less than or equal to the threshold wavelength by reducing the amount of light having a wavelength that is less than or equal to the threshold wavelength by about 50%, or about 80%, or about 90%, or about 5%, or about 99%, or about 99.5%, or about 99.9%. In this way, the light filter may be specified and designed to facilitate use of the oximetry sensor in a particular environment, such as a hospital (e.g., based on a respective wavelength and/or other characteristics of ambient light in the particular environment, which may be estimated, modeled, measured, and so forth, and/or based on a respective wavelength of the light emitted by the light source of the oximetry sensor). Indeed, different oximetry sensors may include different light filters (e.g., specified and designed to filter light according to different threshold wavelengths; provided as part of a kit or suite of oximetry sensors) based on expected use in different environments.

In examples described herein, a method of manufacturing a patient monitoring sensor (e.g., a pulse oximetry sensor) includes attaching a light source to a sensor patch that is configured to be applied to a region of skin of a patient, the light source being configured to emit light having a wavelength greater than the threshold wavelength to, or into, tissue of the patient. The method also includes attaching a detector to the sensor patch, the detector configured to detect filtered light that is indicative of an oxygen saturation level of the patient. The method also includes disposing a light filter to cover the at least the portion of the active area of the detector, the light filter configured to receive the first light from the tissue of the patient (e.g., that was originally from the light source) and ambient light, filter the received light and ambient light based on wavelength, and output the filtered light to the detector.

The example devices and techniques disclosed herein provide improved patient monitoring performance, enable reliable use of an oximetry sensor in a larger range of environments (e.g., including high ambient light environments), and provide an oximetry sensor with a smaller area, or a smaller footprint, that may be used in with larger range of patients or regions of a patient (e.g., on premature infants). The example devices and techniques also improve the case of use of oximetry sensors, for example, by reducing or eliminating the need for a user or clinician to use alternatives or workarounds to mitigate the effects of ambient light. Additionally, the example devices and techniques may improve the case of use of an oximetry sensor by reducing the sensitivity of the sensor to how well the sensor is applied to the patient, e.g., such as on a finger of a patient which may have gaps that may provide a path for ambient light (e.g., as stray light) to the detector. Such stray, ambient light may be filtered out rather than having to carefully eliminate or reduce the gaps.

While the present disclosure describes devices and techniques with reference to an oximetry sensor patch including a light filter, any other suitable sensor configuration including a light filter may be used according to the present disclosure. For example, instead of patches, sensors may be secured to or carried on or within housings, rigid substrates, flexible substrates, bands, rods, or any other sensor support structures or devices.

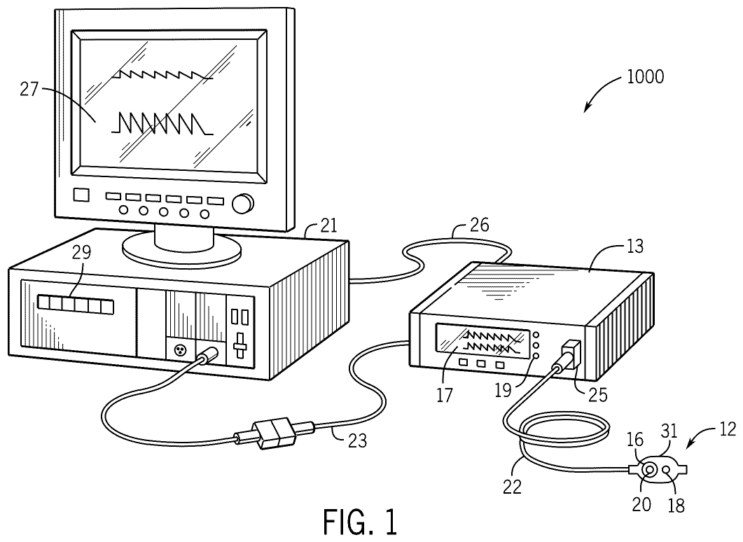

FIG. 1 a perspective view of an example system 1000 configured to monitor one or more physiological parameters of a patient, including an example pulse oximetry sensor patch 12. Pulse oximetry sensor patch 12 may be a forehead patch configured to be applied to a region of skin of a patient, e.g., a forehead of a patient.

Pulse oximetry sensor patch 12 may represent a MAXFAST™, INVOS™, or other optical sensor or oximetry sensor available from Medtronic, Inc. While the present disclosure describes sensors for use on a patient's forehead and/or temple, the pulse oximetry sensor patch 12 may be configured for use on other tissue locations, such as the finger, the toes, the heel, the car, or any other appropriate measurement site. While the system 1000 illustrated in FIG. 1 relates to photoplethysmography or pulse oximetry, the system 1000 may be configured to obtain a variety of medical measurements with a suitable medical sensor. For example, the system 1000 may additionally or alternatively be configured to perform regional oximetry, determine patient electroencephalography (e.g., a bispectral index), or any other physiological parameter.

The system 1000 includes the pulse oximetry sensor patch 12, which may be communicatively coupled to a patient monitor 13. The pulse oximetry sensor patch 12 may include one or more emitters 18 (also referred to herein as the “emitter 18” to facilitate discussion) and one or more detectors 20 (also referred to herein as the “detector 20” to facilitate discussion). The pulse oximetry sensor patch 12 may be coupled to the patient monitor 13 via a cable 22. The cable 22 is configured to interface with the patient monitor 13 through a connector 25, which is adapted to couple to a sensor port of the patient monitor 13. The cable 22 may include a plurality of conductors, such as a first set for each emitter 18 and a second set for each detector 20, which are configured to carry signals (e.g., electrical signals, optical signals) between the patient monitor 13 and pulse oximetry sensor patch 12. The conductors may be surrounded by an insulting material, such that the cable 22 is a rounded cable. Alternatively, the cable 22 may be a ribbon cable or a flexible circuit cable having a relatively flat profile.

The patient monitor 13 may include a monitor display 17 configured to display information relating to one or more physiological parameters of the patient, information about the system 1000, and/or alarm indications. The patient monitor 13 may include various input components 19, such as knobs, switches, keys and keypads, buttons, etc., to provide for operation and configuration of the patient monitor 13. The patient monitor 13 also includes a processor that may be used to execute code such as code for performing diagnostics on the system 1000, for measuring and analyzing patient physiological parameters, and so forth.

The patient monitor 13 may be any suitable monitor, such as a pulse oximetry monitor available from Medtronic, Inc. Furthermore, for providing additional functions, the patient monitor 13 may be coupled to a multi-parameter patient monitor 21 via a cable 23 connected to a sensor input port or via a cable 26 connected to a digital communication port. In addition to the patient monitor 13, or alternatively, the multi-parameter patient monitor 21 may be configured to calculate physiological parameters and to provide a central display 27 for the visualization of information from the patient monitor 13 and from other medical monitoring devices or systems. The multi-parameter monitor 21 includes a processor that may be configured to execute code. The multi-parameter monitor 21 may also include various input components 29, such as knobs, switches, keys and keypads, buttons, etc., to provide for operation and configuration of the multi-parameter monitor 21. In addition, the patient monitor 13 and/or the multi-parameter monitor 21 may be connected to a network to enable the sharing of information, such as patient physiological data captured by the pulse oximetry sensor patch 12, with servers or other workstations.

The pulse oximetry sensor patch 12 may include a sensor body 31 housing one or more optical components. The sensor body 31 may be formed from any suitable material, including rigid or conformable materials, such as foam or other padding materials (e.g., a sponge or gel), fiber, fabric, paper, rubber or elastomeric compositions (including acrylic elastomers, polyimide, silicones, silicone rubber, celluloid, PMDS elastomer, polyurethane, polypropylene, polyethylene, acrylics, nitrile, PVC films, acetates, and latex). In some examples, the pulse oximetry sensor patch 12 may be a bandage, e.g., the sensor body 31 may include or be formed as a sensor bandage.

The sensor body 31 may house a number of components, each providing certain functionality. For example, the pulse oximetry sensor patch 12 may be a wireless sensor. In such examples, the pulse oximetry sensor patch 12 may include a wireless module for establishing a wireless communication with the patient monitor 13 and/or the multi-parameter patient monitor 21 using any suitable wireless standard. By way of example, the wireless module may be capable of communicating using one or more of the ZigBee® standard, WirelessHART® standard, Bluetooth® standard, IEEE 802.11x standards, or MiWi™ standard.

The emitter 18 and the detector 20 may be arranged in a reflectance or transmission-type configuration with respect to one another. For example, when configured for use on a patient's forehead, the emitter 18 and the detector 20 may be in a reflectance configuration. Light from the emitter 18 may be used to measure, for example, oxygen saturation, water fractions, hematocrit, or other parameters of a patient. The term “light” may refer to electromagnetic radiation, e.g., one or more of ultrasound, radio, microwave, millimeter wave, infrared, visible, ultraviolet, gamma ray or X-ray electromagnetic radiation, and may also include any wavelength within the radio, microwave, infrared, visible, ultraviolet, or X-ray spectra, and that any suitable wavelength of light may be appropriate for use with the present disclosure.

In some examples, the emitter 18 may include a light source configured to emit light to, or into, tissue of a patient, the light including a wavelength greater than a threshold wavelength. For example, the emitter 18 may be configured to emit light having red or near infrared (NIR) light, e.g., light greater than about 550 nanometers (nm), or greater than about 600 nm, or greater than about 650 nm, or greater than about 660 nm.

In some examples, the detector 20 may include an array of detector elements that may be capable of detecting light at various intensities and wavelengths. Light may enter the detector 20 after passing through the tissue of the patient. Alternatively, light emitted from the emitter 18 may be reflected by elements in the tissue of the patient to enter the detector 20. The detector 20 may convert the received light at a given intensity, which may be directly related to the absorbance and/or reflectance of light in the tissue of the patient, into an electrical signal. That is, when more light at a certain wavelength is absorbed, less light of that wavelength is typically received from the tissue by the detector 20, and when more light at a certain wavelength is reflected, more light of that wavelength is typically received from the tissue by the detector 20. After converting the received light to an electrical signal, the detector 20 may send the signal to the patient monitor 13, where physiological characteristics may be calculated based at least in part on the absorption and/or reflection of light by the tissue of the patient.

In the example shown, the system 1000 includes a light filter 16. The light filter 16 may be configured to receive light from the emitter 18 and ambient light, filter out at least a portion of the ambient light (e.g., reduce or prevent a portion of ambient light from being output to the detector 20), and output filtered light to the detector 20, the filtered light including at least a portion of the light emitted by the emitter 18. The detector 20 may be configured to detect and/or receive the filtered light, and the filtered light may be indicative of an oxygen saturation level of the tissue and/or the patient. In some examples, the detector 20 is configured to receive filtered light indicative of an oxygen saturation level of the tissue, and is also indicative of an oxygen saturation level of the patient, e.g., the oxygen saturation level of the patient may at least be inferred from the oxygen saturation level of the tissue measured and/or determined by the system 1000.

In some examples, the light filter 16 may be configured to filter light based on wavelength. For example, the emitter 18 may be configured to emit a first light to, or into, tissue of a patient, the first light including a first light wavelength that is greater than a threshold wavelength. The light filter 16 may be configured to receive the first light from the tissue of the patient, and filter a second light wavelength from the received first light (e.g., from the emitter 18 by way of tissue of the patient) or from ambient light, where the second light wavelength is less than or equal to the threshold wavelength. For example, the light filter 16 may filter out, or block, or absorb, or reflect, or redirect, or otherwise reduce or prevent a portion of the second light wavelength from reaching the detector 20. The light filter 16 may be configured to output filtered light that includes at least a portion of the received first light including the first light wavelength from the tissue of the patient, and that does not include, or includes in a reduced amount, the second light wavelength (e.g., light including the second light wavelength; ambient light).

In some examples, the light filter 16 may be an absorption filter, a reflecting filter, an interference filter, a spectral mirror, a diffraction grating, a diffractive optical element, a holographic optical element, or any filter suitable to receive light and output the filtered light to the detector 20. In some examples, the light filter 16 may be a “longpass” absorption filter configured to receive and transmit at least a portion of light having wavelengths greater than the threshold wavelength and to absorb at least a portion of light having wavelengths less than or equal to the threshold wavelength. For example, the light filter 16 may include a light absorbing dye disposed on, or within, a material of the light filter 16. The light filter 16 may include a dye disposed onto or within a polymer material, a polycarbonate, a polymethylmethacrylate (PMMA), an acrylic, a polyurethane, a plastic, a silicone, a glass, or the like. In other examples, the light filter 16 may be a “longpass” interference filter configured to receive and transmit at least a portion of light having wavelengths greater than the threshold wavelength and to reflect at least a portion of light having wavelengths less than or equal to the threshold wavelength, e.g., away from the detector 20. In other examples, the light filter 16 may be a “bandpass” absorption and/or interference filter configured to receive and transmit at least a portion of light having wavelengths greater than a first threshold wavelength and less than a second threshold wavelength that is greater than the first threshold wavelength, and to absorb, reflect, block, or otherwise reduce or prevent transmission of at least a portion of light having wavelengths less than or equal to the first threshold wavelength and/or greater than or equal to the second threshold wavelength.

In some examples, the light filter 16 may have certain characteristics that determine how the light filter 16 filters light. For example, the light filter 16 may have a threshold wavelength for which the light filter 16 passes light having wavelengths greater than the threshold by a particular transmission amount, e.g., a “pass” transmission that is greater than about 50%, or about 85%, or about 90%, or about 95%, or about 99%. The light filter 16 may have a “cut on” wavelength for which the light filter 16 filters (e.g., or blocks, absorbs, reflects, redirects, reduces, or otherwise prevents from reaching the detector 20) light having wavelengths less than or equal to the cut on wavelength by a particular amount, for example by about 50%, or about 85%, or about 90%, or about 95%, or about 99%, or about 99.5%, or about 99.9%. In other words, for wavelengths less than or equal to the cut on wavelength, the light filter 16 may have a “block” transmission of less than about 50%, or less than about 20%, or less than about 10%, or less than about 5%, or less than about 1%, or less than about 0.5%, or less than about 0.1%. For wavelengths of light between the threshold wavelength and the cut on wavelength, the light filter 16 may have a transmission that is between the pass transmission and the block transmission.

In some examples, the light filter 16 may be configured to have a threshold wavelength of about 550 nm, or about 600 nm, or about 650 nm, or about 660 nm. For example, the light emitter 18 may include one or more LEDs configured to emit red and/or NIR light, e.g., light greater than 600 nm, or greater than 650 nm, or light greater than 660 nm, for interacting with tissue of the patient, the detection of which by the detector 20 may be indicative of an oxygen saturation level of the tissue and/or patient. The light filter 16 may be configured to pass the red and NIR light, and to filter out shorter wavelength light.

In some examples, the light filter 16 is configured to cover the entirety of the active area of detector 20, e.g., the area of the detector 20 configured to receive light and convert the received light into an electrical signal indicative of the amount of light received. In other examples, the light filter 16 may be configured to cover a portion of the active area of the detector 20. In some examples, the light filter 16 is configured to cover at least a portion of, or the entirety of, the active area of the detector 20 by being disposed between the detector and both of the tissue of the patient and an ambient light source. In other words, the light filter 16 may cover the detector 20 by being disposed such that light from the tissue of the patient and/or ambient light must be received by the light filter 16 before such light can be incident on the detector 20.

In some examples, the light filter 16 is disposed on the surface of the active area of the detector 20, in whole or in part. For example, the light filter 16 may be laminated to (e.g., via an adhesive), or otherwise attached to (e.g., via a press fit, magnetic attachment, electrostatic attachment, or any suitable attachment means) a surface of the active area of the detector 20, e.g., such that at least a portion of the light filter 16 is in direct contact with at least a portion of a surface of the detector 20 corresponding to the active area of the detector 20. The active area of the detector 20 may include a surface area of the detector 20 that is configured to receive light, e.g., convert light into an electrical signal indicative of the amount of light received. In some examples, the detector 20 may have one or more layers disposed over the active area, e.g., protective coatings. In such cases, the light filter 16 may cover at least a portion of the detector 20 by covering, laminating to, attaching to, or otherwise being disposed on or in contact with an outer surface of the detector 20, such as a protective coating, corresponding to the active area of the detector 20, such that light received by a portion of the active area of the detector 20 is first be received by at least a portion of the light filter 16 and output from the light filter 16 to the detector 20 (e.g., light is blocked from reaching the detector 20 prior to being filtered by the light filter 16; only filtered light reaches the detector 20, and unfiltered light does not reach the detector 20). In some examples, the light filter 16 may encapsulate the active area of the detector 20, e.g., the light filter 16 may be molded over at least a portion of the active area of the detector 20.

In some examples, the light filter 16 may be configured to cover a portion of, or the entirety of, the active area of the detector 20 by first being attached to the sensor body 31, e.g., a sensor patch and/or sensor bandage. For example, an adhesive may be laminated or coated onto the sensor body 31 or a surface of the light filter 16, and the light filter 16 may be attached to or laminated to the sensor body 31 via the adhesive. The detector 20 may then be disposed onto, or attached to, the sensor body 31 with its active area facing light filter 16. In some examples, the light filter 16 may be attached to the sensor body 31, and the detector 20 may be laminated to the light filter 16 via the adhesive, e.g., with the active area of the detector 20 facing the light filter 16. For example, an optical clear adhesive (OCA) may be used to laminate the light filter 16 to the detector 20 and/or the sensor body 31. For example, the light filter 16 may be laminated to the sensor body 31 via an OCA, and the detector 20 may be laminated and/or adhered to the light filter 16 via another OCA, with the active area of detector 20 facing the light filter 16.

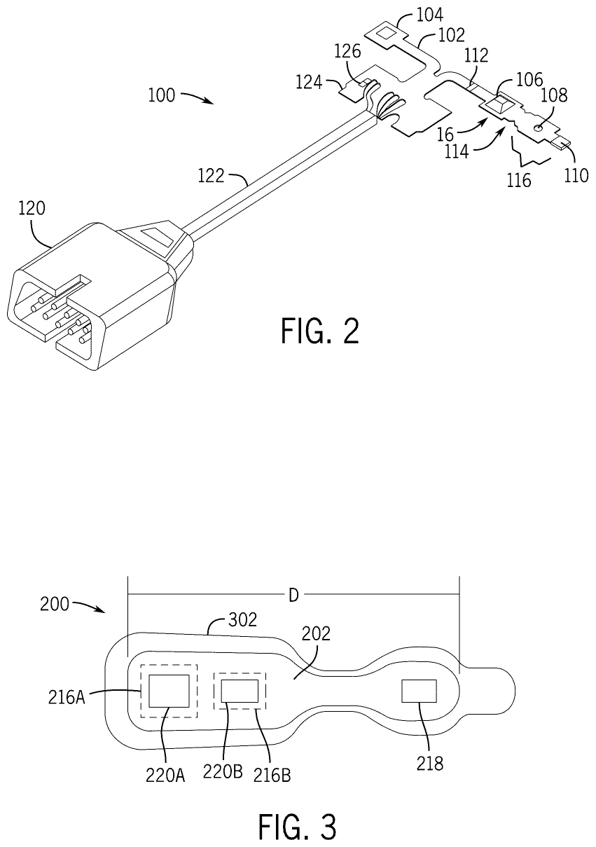

FIG. 2 is a perspective view of an example patient monitoring sensor 100. In some examples, the patient monitoring sensor 100 may be a pulse oximetry sensor, and in some examples, the shape or profile of various components of the patient monitoring sensor 100 may vary. The patient monitoring sensor 100 may be an example of the pulse oximetry sensor patch 12 described herein.

In the example shown, the patient monitoring sensor 100 includes a body 102 that includes a flexible circuit. The patient monitoring sensor 100 includes an emitter 104 (e.g., a surface mount LED) and a detector 106 disposed on the body 102 of the patient monitoring sensor 100.

While any number of exemplary sensor designs are contemplated herein, in the example shown, the body 102 includes a flap portion 116 that includes an aperture 108. The flap portion 116 is configured to be folded at a hinge portion 114 such that the aperture 108 overlaps the detector 106 to allow light to pass through. In some examples, the flap portion 116 includes an adhesive 110 that is used to secure the flap portion 116 to the body 102 after the flap portion 116 is folded at the hinge portion 114.

The patient monitoring sensor 100 includes a plug 120 that is configured to be connected to a patient monitoring system, such as the plug shown connecting the cable 23 to the multi-parameter patient monitor 21 of FIG. 1. The patient monitoring sensor 100 also includes a cable 122 that connects the plug 120 to the body 102 of the patient monitoring sensor 100. The cable 122 includes a plurality of wires 124 that connect various parts of the plug 120 to terminals 126 disposed on the body 102. The flexible circuit is disposed in the body 102 and may be configured to connect the terminals 126 to the emitter 104 and the detector 106. In addition, one of the terminals 126 may connect a ground wire to the flexible circuit.

In some examples, the aperture 108 is configured to provide electrical shielding to the detector 106. In some examples, the aperture 108 also limits the amount of light that is received by the detector 106, e.g., to prevent saturation of the detector 106. In some examples, the aperture 108 may be an aperture of the material of the body 102, but may not be a through hole, e.g., the aperture 108 may be an optically clear aperture that includes a material or component with a substantially high transmission for light having certain wavelengths, such as the light filter 16. In the example shown, the aperture 108 is a through hole and clear aperture, and the light filter 16 is disposed on a surface of the detector 106 to cover an active area of the detector 106. In some examples, the configuration of the aperture 108, e.g., a number, shape, and size of the openings that define the aperture 108, may vary. In the example shown, the aperture 108 includes a single round opening. In other embodiments, the aperture 108 can include one or more openings that have various shapes and sizes. The configuration of the aperture 108 may be selected to provide electrical shielding for the detector 106, and/or control the amount of light that is received by the detector 106, e.g., light from tissue of the patient (signal light) and/or ambient light. In exemplary embodiments, the body 102 includes a visual indicator 112 that is used to assure proper alignment of the flap portion 116 when folded at the hinge portion 114. Further, the shape of the material of the flap portion 116 around the aperture 108 can vary, while at the same time increasing the surface area around the detector 106 to reduce the contact pressure from the detector 106 on skin and/or tissue of the patient.

FIG. 3 is a top view of another example patient monitoring sensor 200. The patient monitoring sensor 200 may be substantially similar to the pulse oximetry sensor patch 12 and/or the patient monitoring sensor 100, except that the patient monitoring sensor 200 may be a regional oximetry sensor, e.g., including one or more emitters 218 (also referred to herein as the “emitter 218” to facilitate discussion) and/or one or more detectors 220 (also referred to herein as the “detector 220” to facilitate discussion) and having a different form factor. In the example shown, FIG. 3 illustrates a portion of the patient monitoring sensor 200 that may be applied to a patient, and other portions, such as wires or plugs that may be a part of the patient monitoring sensor 200, are not shown. The patient monitoring sensor 200 may be used with the system 1000, e.g., the patient monitoring sensor 200 may be configured to be communicatively coupled to the patient monitor 13 (FIG. 1).

In the example shown, the patient monitoring sensor 200 includes the body 202, the emitter 218, the detectors 220A and 220B, and the light filters 216A and 216B disposed over the detectors 220A and 220B so as to cover the active areas of the detectors 220A and 220B, respectively. The body 202 may be an example of the sensor body 31 or the body 102, the emitter 218 may be an example of the emitter 18 or the emitter 104, the detectors 220A and 220B may be examples of the detector 20 and the detector 106, and the light filters 216A and 216B may each be examples of the light filter 16. In some examples, the body 202 may be a bandage that may or may not include flexible circuitry. For example, the body 202 may include one or more layers substantially similar to any or all of layers 402-410 of the patient monitoring sensor 400 described below. Although shown with one emitter 218, the patient monitoring sensor 200 may include more than one emitter 218, e.g., two or more emitters 218.

For example, in FIG. 3, the patient monitoring sensor 200 is shown overlaid with an outline of a larger patient monitoring sensor that does not have a light filter, the outline of the area or “footprint” of the body of which is indicated by outline 302. For example, the light filter 216A and/or the light filter 216B may enable a reduction in the overall size of the sensor body 202 relative to a patient monitoring sensor that does not have a light filter (e.g., relative to the larger patient monitoring sensor indicated by the outline 302). The light filter 216A and/or the light filter 216B may reduce ambient light that would otherwise be absorbed, blocked, reflected, diffused, redirected, or otherwise be reduced at the detectors 220A and 220B by the material of a relatively larger patient monitoring sensor, such as the larger patient monitoring sensor indicated by the outline 302. In the example shown, the body 202 may include a sensor patch having a maximum dimension D (e.g., length, such as length along an axis that extends through the emitter 218 and the detectors 220A and 220B) less than 100 millimeters (mm), or less than 75 mm, or less than 50 mm, or less than 40 mm. In some examples, the sensor body 202 may be reduced in size, e.g., by about 30%, at least in the maximum dimension D, relative to a sensor body of a relatively larger patient monitoring sensor without a light filter, which may be about 69 mm long. For example, the sensor body 202, due to including the light filters 216A and 216B, may be reduced at least in the maximum dimension D from about 69 mm (for a relatively larger patient monitoring sensor without the light filters 216A and 216B) to about 48 mm (for the patient monitoring sensor 200 including the light filters 216A and 216B). In some examples, the sensor body 202 may have the maximum dimension D of less than or equal to about 46 mm.

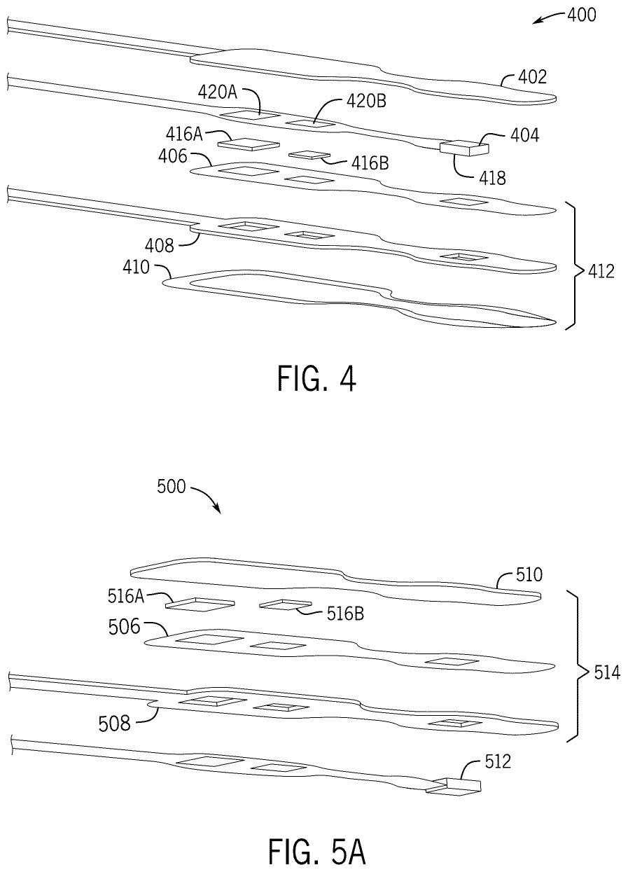

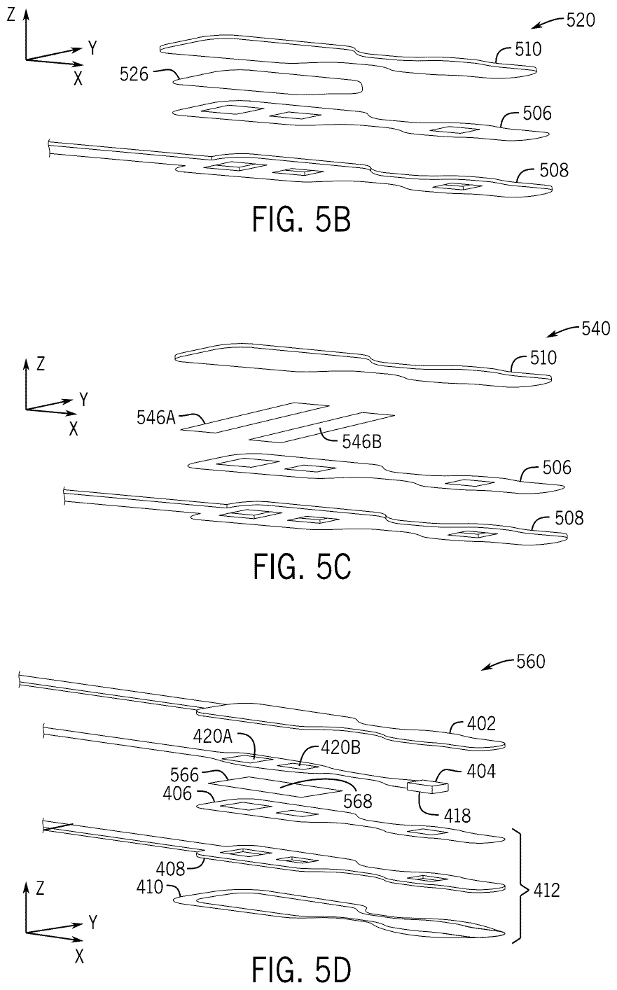

FIG. 4 is an exploded perspective view of another example patient monitoring sensor 400, and FIGS. 5A-5C include exploded perspective views of other example patient monitoring sensors 500, 520, and 540, respectively. The patient monitoring sensors 400, 500, 520, and 540 may be examples of the patient monitoring sensor 200, and illustrate two non-limiting examples of layers of construction of patient monitoring sensors disclosed herein. FIG. 4 illustrates the patient monitoring sensor 400 in which light filters 416A, 416B are separate components that are added during the manufacture or construction of the patient monitoring sensor 400, and FIG. 5A illustrates the patient monitoring sensor 500 in which light filters 516A, 516B are incorporated into a layer of the patient monitoring sensor 500 before assembly and/or before converting the layers of the patient monitoring sensor 500 for assembly. FIGS. 5B and 5C illustrate the patient monitoring sensors 520 and 540, respectively, in which different form factors of light filters are incorporated into a layer of their respective patient monitoring sensors 520, 540 before assembly and/or before converting.

Referring to FIG. 4, the patient monitoring sensor 400 includes an upper layer 402, a flex circuit layer 404, a light blocking layer 406, a bottom layer 408, and a patient adhesive layer 410 (e.g., patient adhesive and/or moisture barrier layer). The light blocking layer 406, the bottom layer 408, and the patient adhesive layer 410 may be a part of a sensor assembly 412 (e.g., sensor package). Indeed, the light blocking layer 406, the bottom layer 408, and the patient adhesive layer 410 may be attached (e.g., coupled) to one another and/or manufactured as the sensor assembly 412. In the example shown, the upper layer 402 may provide structural support and flexible cushioning and/or protection for the patient monitoring sensor 400. The flex circuit layer 404 is configured to electrically and communicatively connect an emitter 418 and detectors 420A and 420B to a patient monitor (not shown), e.g., the patient monitor 13 (FIG. 1). The emitter 418 may be an example of the emitters 218, 104, and/or 18 described herein. The detectors 420A and 420B may be examples of the detectors 220A, 220B, 106, and/or 20 described herein.

In the example shown, the emitter 418 and the detectors 420A and 420B are attached to, and electrically connected to, one or more conductors of the flex circuit layer 404. The light blocking layer 406 may be configured to absorb, reflect, block, or otherwise prevent and/or reduce light propagating to the detectors 420A, 420B through the patient monitoring sensor 400, e.g., to prevent light shunting, from the emitter 418 and/or from ambient light. In some examples, the light blocking layer 406 may be configured to provide electromagnetic shielding, e.g., the light blocking layer 406 may include one or more conductors. The bottom layer 408 may be configured to provide cushioning, padding, or the like for the patient, and/or an offset between the emitter 418 and the detectors 420A and 420B so as to improve patient comfort during use of the patient monitoring sensor 400. For example, the bottom layer 408 may include a foam and/or thermal insulating material to provide a substantially soft and flexible layer that may also provide thermal insulation configured to reduce heat transfer to the skin of the patient (e.g., heat from the emitter 18).

In some examples, the bottom layer 408 may have a thickness configured to reduce and/or eliminate the patient being able to feel the emitter 418 and the detectors 420A and 420B protruding towards skin of the patient. The patient adhesive layer 410 may be configured to adhere the patient monitoring sensor 400 to skin of the patient with an emitting surface of the emitter 418 and the active areas of detectors 420A, 420B facing the skin of the patient. In some examples, the patient adhesive layer 410 may include a removable liner configured to protect the adhesive when not in use, and to be removable for application of patient monitoring sensor to the patient. In some examples, the patient adhesive layer 410 may be removable and repositionable, e.g., removable from, and repositionable and/or replaceable onto, the skin of the patient.

In the example shown, the light blocking layer 406 and the bottom layer 408 each include apertures corresponding to the emitter 418 and the detectors 420A, 420B. In some examples, the apertures are holes without any material, and in other examples, the apertures are optically clear apertures that include a material with a high transmissivity for light having wavelengths emitted by the emitter 418 and/or detectable by the detectors 420A, 420B.

In the example shown, the patient monitoring sensor 400 includes the light filters 416A and 416B. The light filters 416A, 416B may be examples of the light filters 216A, 216B, and/or 16 described herein. As described herein, the sensor assembly 412 may include the light blocking layer 406, the bottom layer 408, and the patient adhesive layer 410. In some embodiments, during assembly of the patient monitoring sensor 400, the light filters 216A and 216B may be installed onto the flex circuit layer 404 (e.g., onto the detector 420A and/or 420B). For example, the light filters 216A and 216B may be attached (e.g., adhered) to the flex circuit layer 404. In this manner, the patient monitoring sensor 400 may then be efficiently assembled by attaching the sensor assembly 412 to the flex circuit layer 404, which includes the light filters 216A and 216B. Further, the sensor assembly 412 and the flex circuit layer 404 may be attached to the upper layer 402.

In the example shown, the light filter 416A is disposed to cover at least a portion of the active area of the detector 420A, and the light filter 416B is disposed to cover at least a portion of the active area of the detector 420B. The light filters 416A, 416B may be an extruded thin-film plastic including one or more absorbing dyes, e.g., mixed with a plastic resin to create thin-film spectral light filters 416A, 416B. In the example shown, the light filters 416A, 416B may be laminated to the detectors 420A, 420B, respectively, e.g., laminated to at least a patient-facing surface of the detectors 420A, 420B. For example, an OCA may be used to laminate the light filters 416A, 416B to the detectors 420A, 420B. In some examples, an OCA may be coated or laminated onto the detectors 420A, 42B, and the light filters 416A, 416B may then be laminated to the detectors 420A, 420B, or an OCA may be coated or laminated onto a surface of the light filters 416A, 416B which may then be laminated to the detectors 420A, 420B. In some examples, the OCA may have an index of refraction substantially the same as the encapsulation layer and/or material of the detectors 420A, 420B.

In some examples, the light filters 416A, 416B may be laminated to the detectors 420A, 420B via a transfer adhesive, which may or may not be an OCA. For example, the light filters 416A, 416B may be laminated to the detectors 420A, 420B using a Clear 1522 tape from 3M™, Inc. In some examples, laminating the light filters 416A, 416B to the detectors 420A, 420B with an adhesive having an index of refraction substantially the same as the encapsulation layer and/or material of the detectors 420A, 420B may reduce reflections at optical interfaces (e.g., between the adhesive and the light filters 416A 416B, and between the adhesive and the detectors 420A, 420B) and may reduce light loss (e.g., light that is filtered through the light filters 416A, 416B to be received by the active areas of the detectors 420A, 420B.

In some examples, the light filters 416A 416B may include a transfer adhesive and may be die-cut to size, e.g., via a converting system, placed to cover the detectors 420A, 420B, respectively, and laminated to (e.g., pressed to adhere to) the detectors 420A, 420B either manually by an operator or manufacturer, or automatically, such as by a pick and place machine.

Referring to FIG. 5A, the patient monitoring sensor 500 includes a bottom layer 508, a light blocking layer 506, a moisture barrier layer 510 (e.g., patient adhesive and/or moisture barrier layer), a flex circuit layer 512, and light filters 516A, 516B. The patient monitoring sensor 500 may also include an upper layer (not shown) that may be substantially the same as the upper layer 402, wherein the flex circuit layer 512 may be positioned or layered between the bottom layer 508 and the upper layer 502. Additionally, the flex circuit layer 512 may support an emitter similar to the emitter 418 and detectors similar to the detectors 420A, 420B described herein. The bottom layer 508, the light blocking layer 506, the moisture barrier layer 510, and the light filters 516A, 516B may each be substantially similar to the bottom layer 408, the light blocking layer 406, the patient adhesive layer 410, and the light filters 416A, 416B described herein. The bottom layer 508, the light blocking layer 506, the light filters 516A and 516B, and the moisture barrier layer 510 may be a part of a sensor assembly 514 (e.g., sensor package). That is, the bottom layer 508, the light blocking layer 506, the light filters 516A and 516B, and the moisture barrier layer 510 may be coupled (e.g., attached) to one another and/or manufactured as the sensor assembly 514. In the example shown, the patient monitoring sensor 500 includes the light blocking layer 506 on the patient-facing side of the bottom layer 508, but in some examples the light blocking layer 506 may be on the opposite side of the bottom layer 508. Additionally, regarding the patient monitoring sensor 400, the light blocking layer 406 is illustrated as being on the non-patient-facing side of the bottom layer 408, but in some examples the light blocking layer 406 may be on the patient-facing side of the bottom layer 408.

In the example shown, the light filters 516A, 516B may be incorporated into one or more of the layers 508, 506, 510, or 512 and registered to cover the detectors of the patient monitoring sensor 500 after assembly. For example, the light filters 516A, 516B may be installed onto the light blocking layer 506, such that the light filters 516A, 516B are positioned between the light blocking layer 506 and the moisture barrier layer 510, as well as aligned with (e.g., covering; extending across) apertures formed in the light blocking layer 506 and/or the bottom layer 508, which are aligned with (e.g., cover) the detectors on the flex circuit layer 512 . . . . In this manner, the patient monitoring sensor 500 may be efficiently assembled by coupling the sensor assembly 514 that includes the light filters 516A, 516B (e.g., laminated between the layers 506, 508, 510 and aligned with the apertures), to the flex circuit layer 512. In some examples, any or all of the layers 506, 508, and 510 may be produced using automation on a converter with feeders, laminating rollers, and die-cutting tools. A film comprising the light filter 516A and/or 516B may be laminated with a transfer adhesive, which would then be die-cut or molded to size. In the example shown, the light filters 516A, 516B are laminated to the non-patient-facing side of the moisture barrier 510 and positioned so as to correspond to the apertures of the layers 506 and 508 and align with (e.g., cover) the detectors of the patient monitoring sensor 500 after assembly (e.g., registered to, or in registration with, the detectors of patient monitoring sensor 500).

Referring to FIG. 5B, the patient monitoring sensor 520 may be substantially similar to the patient monitoring sensor 500 of FIG. 5A, except that the patient monitoring sensor 520 includes light filter 526. The light filter 526 may be sized to as to correspond to both of the apertures of the layers 506 and 508 and cover detectors of the patient monitoring sensor 520 after assembly, e.g., without the need for registration a single light filter disposed over at least a portion of the area of the moisture barrier 510 and covering the apertures of the layers 506 and 508 and the detectors of patient monitoring sensor 520 after assembly.

Referring to FIG. 5C, the patient monitoring sensor 540 may be substantially similar to the patient monitoring sensor 500 of FIG. 5A, except that patient monitoring sensor 540 includes light filters 546A and 546B. The light filters 546A and 546B may be substantially similar to the light filters 516A and 516B, except that the light filters 546A and 546B may be converted as lanes, e.g., two lanes along the y-direction and separated in the x-direction as shown. For example, the light filters 546A and 546B may be converted in a machine direction of a converting process and assembled across a plurality of patient monitoring sensors 540 (arranged in the machine direction) as strips that are converted upon converting each individual patient monitoring sensors 540 after assembly. In this way, the light filters 546A and 546 may be efficiently incorporated into multiple sensor assemblies, which may then be coupled to respective flex circuits to form multiple patient monitoring sensors. In FIGS. 5B and 5C, it should be appreciated that the layers 506, 508, 510 with the light filters 546A and 546B may form a sensor assembly (e.g., laminated together to form the sensor assembly, as described herein), and the sensor assembly may be coupled to a flex circuit that includes an emitter and detectors (e.g., as shown in FIG. 5A).

Referring to FIG. 5D, a patient monitoring sensor 560 may be substantially similar to the patient monitoring sensor 400 of FIG. 4, except that the patient monitoring sensor 560 includes a light filter 566 and alignment features 568. The light filter 566 may substantially similar to the light filters 526, except that the light filter 566 may be configured to cover the plurality of detectors 420A, 420B without covering a substantial amount of non-detector area, e.g., to cover only the detectors 420A, 420B and as little non-detector area as is feasible with a one-piece material and/or to adhere to the detectors 420A, 420B and/or to adhere to the flex circuit 404 to cover the detectors 420A, 420B (or alternatively to adhere to the light blocking layer 406 or other layer 408, 410 as part of the sensor assembly 412. In some examples, the light filter 566 is configured to improve the flexibility of the patient monitoring sensor 560 (or alternatively reduce the stiffness of the patient monitoring sensor 560), e.g., relative to the patient monitoring sensor 520 of FIG. 5B, by reducing the amount of material used in the light filter 566 (e.g., area, volume, thickness) in the assembled patient monitoring sensor 560. In the example shown, the light filter 566 extends in the x-direction so as to cover the detectors 420A, 420B in the x-direction, and the light filter 566 extends in the y-directions so as to cover detectors 420A, 420B in the y-direction, and covers the non-detector area between the detectors 420A, 420B, and in some examples, a small portion of a border area around the detectors 420A, 420B (e.g., extends beyond respective edges of the detectors 420A, 420B in the x-direction and/or the y-direction by less than 5, 4, 3, 2, or 1 mm; extends beyond the respective edges in this manner, and also optionally to cover the non-detector area between the detectors 420A, 420B. as shown). In the example shown, the light filter 566 includes the alignment feature 568. The alignment feature 568 may be configured to align the light filter 566 positionally (e.g., in the x-y directions) and rotationally (e.g., about the z-axis as shown so as to correctly orient the light filter 566), e.g., during assembly. In the example shown, the alignment feature 568 includes a “D-shaped” pinhole which may be aligned by a fixture having a similarly D-shaped pin, e.g., to register (or positionally and rotationally align) the light filter 566 so as to cover the detectors 420A, 420B when assembled. In other examples, alignment features 568 may have any suitable shape, e.g., a torx hole, an ellipse, a circle, and alignment features 568 may be positioned in other locations on the light filter 566. In some examples, the alignment feature 568 may include a plurality of alignment features. For example, the alignment feature 568 may include two or more D-shaped holes (or any other suitable shaped hole) that are spaced apart from each other.

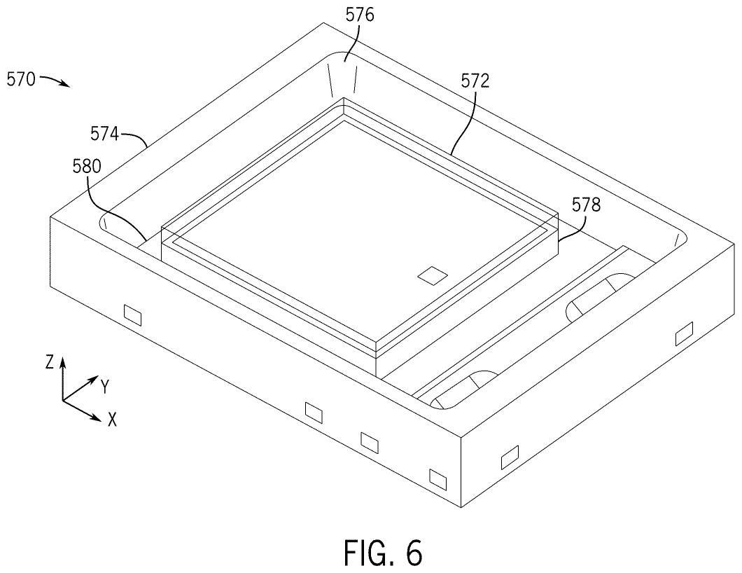

FIG. 6 is a perspective view of an example detector package 570 that includes a light filter 572. The detector package 570 may be employed in various (e.g., different) configurations. For example, the detector package 570 may be employed in the patient monitoring sensor 400 of FIG. 4. Indeed, as an example, two detector packages 570 may be employed in place of the detectors 420A and 420B and the light filters 216A and 216B, which are coupled to the flex circuit layer 404 of FIG. 4 (e.g., one detector package 570 in place of the detector 420A and the light filter 216A, and another detector package 570 in place of the detector 420B and the light filter 216B). The detector package 570 may include a rim 574 (e.g., component package rim; frame), potting material 576, a detector die 578, and/or a package base 580. To facilitate discussion, the detector package 570 and its components may be described with reference to x, y, and z axes, as shown.

During manufacture, the light filter 572 may be coupled to a surface of the detector die 578 (e.g., on a patient-facing side of the detector die 578), which may include an active area of a detector to enable detection of light by the detector. For example, an amount of adhesive (e.g., glue) may be applied to the surface of the detector die 578 and the light filter 572 may be positioned (e.g., placed) on top of the glue. It should be noted that the light filter 572 may be coupled to the detector die 578 using any suitable adhesive. Further, the glue may be cured after the light filter 572 is applied to the detector die 578. In some embodiments, the light filter 572 may include a thickness of equal to or less than 20, 25, 30, or 35 millimeters. Further, in some embodiments, the light filter 572 may be positioned within the detector package 570 such that a gap is maintained between the light filter 572 and a surface of the rim 574 of the detector package 570 relative to the z-axis (e.g., the surface of the light filter 572 is recessed within the rim 574 of the detector package 570 and offset relative to the surface of the rim 574 along the z-axis). For example, the gap may be equal to or greater than approximately 2, 3, 4, 5, or 6 millimeters along the z-axis. In this manner, the gap may enable accommodation of the potting material 576 within the rim 574 and over the light filter 572 (e.g., to cover the light filter 572; extend over and cover a patient-facing side of the light filter 572) without causing protrusion of components of the detector package 570 (e.g., beyond the rim 574 of the detector package 570). Moreover, in some embodiments, the light filter 572 may include any suitable color or tint. For example, the light filter 572 may be a red filter.

The rim 574 may include or define an outer edge or a border of the detector package 570, which may protect and/or hold the components (e.g., the light filter 572, the detector die 578) of the detector package 570. During manufacture, after placement of the light filter 572 on the detector die 578, the potting material 576 may be dispensed (e.g., disposed) within an internal volume of the detector package 570 at a particular volume (e.g., on the package base 580 and within the rim 574 to cover the light filter 572 and the detector die 578) and cured for a particular duration (e.g., 10 minutes, 20 minutes, 30 minutes, and so on). In some embodiments, the potting material 576 may include plastic and/or silicone. The potting material 576 may include a transparent material that may encapsulate and/or protect the components of the detector package 570. The package base 580 may include features for mounting the detector die 578 to the detector package 570, features for mounting the detector package 570 to a flex circuit (e.g., the flex circuit 404 of FIG. 4), and/or provide structural support for the detector package 570.

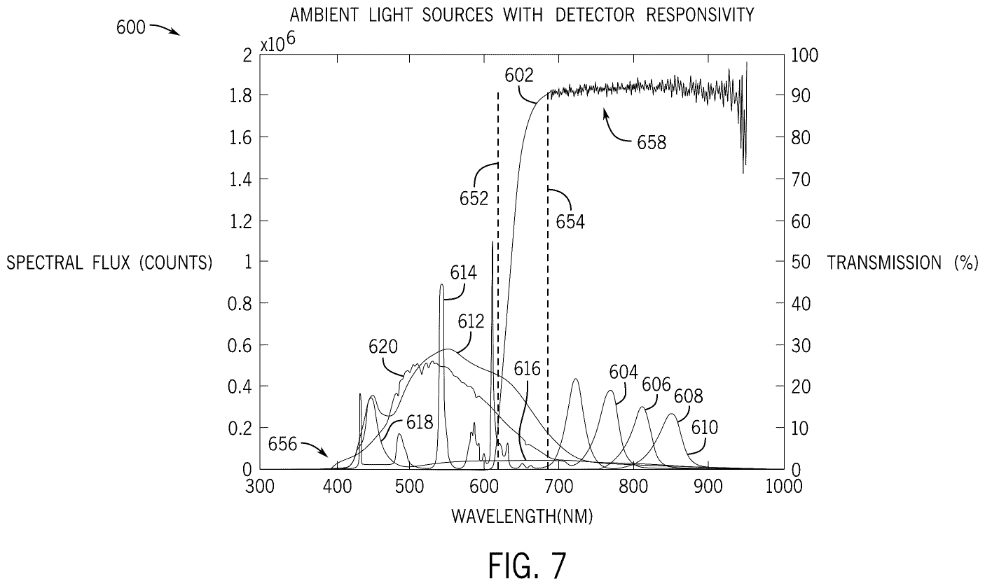

FIG. 7 is a graph 600 including a plurality of plots 602-620 illustrating the spectral output of a plurality of light sources (plots 604-620) overplotted with a spectral transmission (plot 602) of an example light filter. In the example shown, plot 602 is a spectral transmission plot of an example light filter, and although is described with reference to the light filter 16, the plot 602 may be an example of the spectral transmission of any of the light filters 16, 216A, 216B, 416A, 416B, 516A, 516B, and/or 572 described herein. Plots 604-610 are plots of the spectral output of four example emitters, each of which may be an example of the emitter 18, 104, 218, and/or 418 described herein.

Plot 612 is a plot of the spectral output of an example white, or broadband LED, e.g., a UV LED including phosphors configured to output light over a broad range of wavelengths. Plot 614 is a plot of the spectral output of an example fluorescent light source, plot 616 is a spectral output of an example halogen light source, plot 618 is a spectral output of an example UV light source (e.g., LED, mercury lamp, BiliSoft™ phototherapy light from General Electric Co., or any suitable UV light source), and plot 620 is an example spectral irradiance of sunlight through windows in a room (e.g., an example blackbody spectral output).

Plots 612-620 are examples of the spectral content of ambient light from a variety of ambient light sources. Any of the patient monitoring systems and/or sensors described herein may be used in an environment including ambient light from multiple sources, e.g., overhead lighting (e.g., white LED, fluorescent, or incandescent), surgical lighting (e.g., bright, white LED light) neonatal warmers (which may include UV light from bilirubin light sources), and sunlight from windows. The ambient light may cause interference with a patient monitoring sensor without a light filter. Too much interference may cause erroneous readings and signal dropouts due to saturation of the front-end electronics.

In some examples, any of the detectors 20, 106, 220A, 220B, 420A, 420B, and/or 578 may have a spectral responsivity to light that includes light wavelengths outside the useful range for measurement of a patient parameter. For example, the detectors 20, 106, 220A, 220B, 420A, 420B, and/or 578 may be silicon detectors having a spectral responsivity from about 200 nm to about 1200 nm, whereas the spectral output of the emitters 18, 104, 218, and/or 418 may utilize only red and NIR light, e.g., from about 660 nm to about 915 nm (shown as plots 604-610). Ambient light, e.g., from sources having spectral content such as shown in plots 612-620, may then be detected by any of the detectors 20, 106, 220A, 220B, 420A, 420B, and/or 578 if such ambient light is allowed to propagate to the active area (e.g., detecting material) of the detectors, e.g., including light outside of the useful range for measurement, such as from about 200 nm to about 660 nm.

In the example shown, plot 602 illustrates some of the optical characteristics, or design parameters, of light filters disclosed herein, e.g., the light filter 16. The characteristics of plot 602 described below serve as description of characteristics of the light filter 16. In some examples, the light filter 16 may be a longpass filter, referring to a relatively high transmission of light comprising longer wavelengths and a relatively low transmission (or high absorption) of light comprising shorter wavelengths. In some examples, the light filter 16 may be a longpass interference filter comprising one or more layers of reflective or semi-reflective materials separated by a gap and/or a transparent material. In the example shown, the plot 602 corresponds to the transmission of a longpass absorption filter. For example, light filter 16 may comprise one or more absorbing dyes configured to absorb some wavelengths of light but not other wavelengths of light. The amount and type of dyes, and the thickness of the light filter 16, may determine the spectral shape of the transmission of the light filter 16, e.g., the shape of the plot 602.

In the example shown, the plot 602 has a “cut on” wavelength 652 of about 680 nm, and a threshold wavelength 654 (alternatively referred to as a “cut off” wavelength) of about 610 nm. For example, the light filter 16 may be configured to filter, e.g., block, absorb, or the like, wavelengths of light that are less than or equal to the threshold wavelength 654. The plot 602 has a “block region” 656 having a transmission that is less than about 1% for wavelengths of light that are less than the cut on wavelength, e.g., less than about 610 nm, and a “pass region” 658 having a transmission that is greater than about 85% for wavelengths of light that are greater than the threshold wavelength, e.g., greater than about 680 nm. In the example shown, the plot 602 has a substantially linear transmission for wavelengths of light between the cut on wavelength 652 and threshold wavelength 654, e.g., with curvature at about the cut on wavelength 652 and threshold wavelength 654. In other examples, the plot 602 may have a transmission having any suitable shape for wavelengths between the cut on wavelength 652 and the threshold wavelength 654.

In some examples, the plot 602 (and the light filter 16) may have different characteristics than those illustrated in FIG. 7, e.g., a different cut on wavelength 652, threshold or cut off wavelength 654, block region 656 transmission, and/or pass region 658 transmission. For example, the plot 602 and the light filter 16 may have a cut on wavelength of about 550 nm, or about 585 nm, or about 600 nm, or about 610 nm, or about 650 nm, or any wavelength greater than or equal to about 550 nm. In some examples, the plot 602 and the light filter 16 may have a threshold (or cut off) wavelength 654 of about 700 nm, or about 675 nm, or about 660 nm, or about 650 nm, or about 600 nm, or about 550 nm, or any wavelength less than or equal to about 750 nm. In some examples, the plot 602 may have a block region 656 transmission that is less than (and filter 16 may be configured to pass less than) about 50%, or less than about 20%, or less than about 10%, or less than about 5%, or less than about 1%, or less than about 0.5%, or less than about 0.1%. For example, and stated alternatively, the light filter 16 may be configured to filter, e.g., block, absorb, reduce, or otherwise prevent from reaching detector 20, about 50%, or about 85%, or about 90%, or about 95%, or about 99%, or about 99.5%, or about 99.9% of the amount of light having wavelengths in block region 656. The plot 602 may have a pass region 658 transmission that is at least 50%, or at least 80%, or at least 85%, or at least 90%, or at least 95%, or at least 99%.

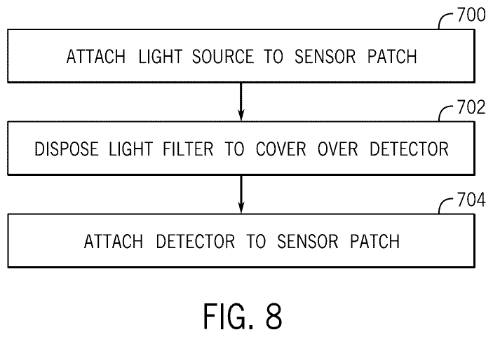

FIG. 8 is a flowchart illustrating an example technique of manufacturing a patient monitoring sensor. While the technique is described with reference to the patient monitoring system 400 and the light filter 416A, the technique may be practiced with any oximetry sensor, pulse oximetry sensor, regional oximetry sensor or optical sensor or light filter according to the present disclosure.

A manufacturer may attach a light source to a sensor patch of an oximetry sensor (700). For example, a manufacturer may solder, adhere, or otherwise attach the emitter 418 to the flex circuit layer 404. In some examples, the light source is configured to emit light into tissue of patient, e.g., light comprising a wavelength greater than a threshold wavelength. For example, the emitter 418 may be configured to emit red and/or NIR light, e.g., light comprising a one or more wavelengths greater than about 550 nm, or greater than about 600 nm, or greater than about 650 nm. In some examples, the manufacturer may assemble the flex circuit layer 404 with the sensor assembly 412 that includes the upper layer 402, the light blocking layer 406, the bottom layer 408, and the patient adhesive layer 410, to form a sensor patch configured to be applied to a region of skin of a patient.

The manufacturer may dispose a light filter to cover over at least a portion of an active area of a detector (702). For examples, the manufacturer may dispose the light filter 416A to cover over at least a portion of the active area of the detector 420A. In some examples, the manufacturer may dispose the light filter 416A to cover over the entirety of the active area of the detector 420A. In some examples, the manufacturer may dispose the light filter 416A onto a surface of the active area of the detector 420A, e.g., the manufacturer may coat or laminate the light filter 416A onto a surface of the detector 420A.

The light filter 416A may be configured to receive light from the tissue of the patient, e.g., the light emitted by the emitter 418 into the tissue of the patient. The light filter 416A may be configured to filter ambient light, e.g., light comprising one or more wavelengths that are less than or equal to the threshold wavelength. For example, the light filter 416A may be configured to filter out, absorb, reflect, redirect, scatter, block, or otherwise reduce or prevent wavelength of light less than or equal to about 660 nm, about 650 nm, about 600 nm, or about 550 nm from being incident on (or received by) the active area of the detector 420A. In some examples, the light filter 416A may receive light from tissue of the patient (that was emitted by the emitter 418) and ambient light, and output filtered light that includes the received light from the tissue, and excludes at least a portion of the received ambient light. In some examples, the light filter 416A may filter received light and output filtered light that includes at least a portion of the received light from the tissue of the patient that was emitted by the emitter 418 but does not include light having a wavelength that is less than or equal to the threshold wavelength.

In some examples, the manufacturer may dispose the light filter 416A directly on the detector 420A. For example, the manufacturer may mix a light absorbing dye with a plastic resin and form (e.g., via molding, extruding, or any suitable technique) the light filter 416A as a thin film filter. The manufacturer may then coat or laminate an adhesive to a side (e.g., a major surface) of the light filter 416A, such as an OCA and/or a transfer adhesive. The manufacturer may die-cut the light filter 416A (or many light filters 416A) to size, e.g., manually or via an automated converting system. The die-cut light filter 416A may then be placed on the detector 420A and laminated to the detector 420A, manually by an operator or automatically, e.g., via an automated pick and place machine. In other examples, the manufacturer may apply or form a thin-film optical coating directly onto a substrate, such as a plastic or glass, to form the light filter 416A, and then dispose the formed light filter 416A on the detector 420A, e.g., via an OCA, a transfer adhesive, or via any suitable means for attaching the light filter 416A to the detector 420A and/or positioning the light filter 416A in the optical path just before the active area of the detector 420A.

The manufacturer may then laminate the light filter 416A to an outer surface of the detector 420A to cover over at least a portion of the active area, or the entire active area, of the detector 420A via the adhesive. In some examples, the manufacturer may laminate the light filter 416A to an outer surface of the detector 420A with an OCA having an optical index of refraction that substantially matches the optical index of refraction of the light filter 416A (e.g., the material of the light filter 416A in which the absorbing dye is included) and/or an optical index of refraction of the detector 420A, e.g., the material (encapsulant, covering, or the like) comprising the outer surface of the detector 420A.

In some examples, the manufacturer may dispose the light filter 416A onto a sensor patch (e.g., one or more of the layers 406, 408, or 410) rather than directly onto the detector 420A. In some examples, the manufacturer may then adhere or attach the detector 420A to the light filter 416A, e.g., during assembly of the layers 402, 404, 406, 408, and 410. In other examples, the manufacturer may assemble the layers 402, 404, 406, 408, and 410 without adhering the detector 420A to the light filter 416A, and the light filter 416A may be disposed to cover over the detector 420A without being attached to the detector 420A, e.g., the light filter 416A may be a “window” for the detector 420A. For example, the manufacturer may laminate the light filter 416A to the patient adhesive layer 410 in registration with (e.g., positioned to correspond to) the detector 420A such that the light filter 416A is disposed to cover over at least a portion, or the entirety of, the active area of the detector 420A.

In some examples, the manufacturer may dispose the light filter 416A to cover at least the portion of the active area of the detector 420A by encapsulating the entire active area of the detector 420A by the light filter 416A. For example, the manufacturer may integrate and/or mix a light absorbing dye within an overmold material and overmold (e.g., via injection molding or any suitable molding technique) the material to form the light filter 416A directly onto the detector 420A to encapsulate the entire active area of the detector 420A. In some examples, the manufacturer may overmold the detector 420A with the light filter 416A before attaching, soldering, and/or electrically connecting the detector 420A to the flex circuit layer 404 (e.g., at method step 706).

In some examples, the manufacturer may dispose the light filter 416A to cover at least the portion of the active area of the detector 420A by integrating the light filter 416A during the manufacture of the detector 420A. For example, the manufacturer may laminate and/or coat the light filter 416A onto a wafer (e.g., a silicon wafer) comprising a plurality of detector dies, dice the plurality of detector dies, and encapsulate the detector dies to form a plurality of detectors 420A with light filter 416A integrated. In some examples, the manufacturer may laminate and/or coat the light filter 416A directly onto the active area or material (e.g., silicon) of the detector 420A. In some examples, the manufacturer may coat the light filter 416A directly onto the detector 420A, e.g., coat or dispose a thin film optical coating onto at least a portion of a surface of the active area of the detector 420A. In some examples, the manufacturer may thin film coat an absorbing dye directly onto the active area (e.g., photosensitive area) or material (e.g., silicon) of the detector 420A to form the light filter 416A integrated with the detector 420A. In some examples, the manufacturer may place a potting material over the light filter 416A that is coupled to the detector 420A, e.g., coat or dispose the potting material in a volume defined by a base and a rim of a detector package to cover the light filter 416A and the detector 420A, as shown and describe with reference to FIG. 6.

The manufacturer may attach the detector 420A to the sensor patch (704). For example, the manufacturer may solder, electrically connect, adhere, laminate, or otherwise attach the detector 420A to the flex circuit layer 404.

The following examples illustrate example subject matter described herein.

Example 1. An oximetry sensor comprising: a light source configured to emit first light into a tissue of a patient, the first light comprising a first light wavelength greater than a threshold wavelength; a light filter configured to: receive the first light from the tissue of the patient; filter a second light wavelength from at least one of the received first light or ambient light, wherein the second light wavelength is less than or equal to the threshold wavelength; and output a filtered light that includes the received first light from the tissue; and a detector configured to detect the filtered light, the filtered light being indicative of an oxygen saturation level of the patient.

Example 2. The oximetry sensor of example 1, wherein the filtered light comprises the first light wavelength and does not comprise the second light wavelength.

Example 3. The oximetry sensor of example 1 or example 2, wherein the oximetry sensor has a form of a sensor patch configured to be applied to a region of skin of the patient.

Example 4. The oximetry sensor of example 3, wherein the sensor patch has a maximum dimension of less than 100 millimeters, or less than 75 millimeters, or less than 50, or less than 40 millimeters.

Example 5. The oximetry sensor of any one of examples 1-4, wherein the threshold wavelength 600 nanometers.

Example 6. The oximetry sensor of any one of examples 1-5, wherein the light filter is configured to cover the entirety of an active area of the detector.

Example 7. The oximetry sensor of example 6, wherein the light filter is disposed on a surface of the active area of the detector, wherein the light filter comprises an alignment feature configured to register the light filter to cover the detector.

Example 8. The oximetry sensor of any one of examples 1-7, wherein the light filter comprises an absorption filter, and the absorption filter comprises a light absorbing dye disposed on or within a plastic material.

Example 9. The oximetry sensor of example 1, further comprising a detector package with the detector, the light filter, and a potting material over the light filter.

Example 10. The oximetry sensor of any one of examples 1-9, wherein the light filter is configured to filter at least 80% of the second light wavelength, or at least 95% of the second light wavelength, or at least 99% of the second light wavelength.

Example 11. The oximetry sensor of any one of examples 1-10, wherein the light filter is configured to: pass at least 80% of the first light wavelength of the received first light from the tissue; and pass less than 5% of a cut-on light wavelength that is less than the threshold wavelength.

Example 12. The oximetry sensor of example 11, wherein the cut-on wavelength is 610 nm, wherein the threshold wavelength is 680 nm.

Example 13. The oximetry sensor of any of examples 1-12 further comprising processing circuitry configured to generate information indicative of the oxygen saturation level based on the detected filtered light.

Example 14. The oximetry sensor of claim 1, further comprising: a flex circuit that supports the light source and the detector; and a sensor assembly comprising a plurality of layers and the light filter laminated between adjacent layers of the plurality of layers, wherein the flex circuit is configured to couple to the sensor assembly to form the oximetry sensor.

Example 15. A method of manufacturing an oximetry sensor, the method comprising: attaching a light source to a sensor patch of the oximetry sensor, wherein the light source is configured to emit first light into a tissue of a patient, the first light comprising a first light wavelength greater than a threshold wavelength, wherein the sensor patch is configured to be applied to a region of skin of a patient; disposing a light filter to cover over at least a portion of an active area of a detector, wherein the light filter is configured to: receive the first light from the tissue of the patient; filter a second light wavelength from at least one of the received first light or ambient light, wherein the second light wavelength is less than or equal to the threshold wavelength; and output a filtered light that includes the received first light from the tissue; and attaching the detector to the sensor patch, wherein the detector is configured to detect the filtered light, the filtered light being indicative of an oxygen saturation level of the patient.

Example 16. The method of manufacturing the patient monitoring sensor of example 15, wherein the filtered light comprises the first light wavelength and does not comprise the second light wavelength.

Example 17. The method of manufacturing the patient monitoring sensor of claim 15 or example 16, wherein the sensor patch has a maximum dimension of less than 100 millimeters, or less than 75 millimeters, or less than 50 millimeters, or less than 40 millimeters.

Example 18. The method of manufacturing the patient monitoring sensor of any one of examples 15-17, wherein the threshold wavelength 600 nanometers.

Example 19. The method of manufacturing the patient monitoring sensor of any one of examples 15-18, wherein disposing the light filter to cover over at least a portion of the active area of the detector comprises covering the entirety of the active area of the detector with the light filter.

Example 20. The method of manufacturing the patient monitoring sensor of any one of examples 15-19, wherein disposing the light filter to cover the at least the portion of the active area of the detector comprises disposing the light filter onto a surface of the active area of the detector.

Example 21. The method of manufacturing the patient monitoring sensor of any one of examples 15-20, wherein the light filter comprises a light absorbing dye disposed on or within a plastic material.

Example 22. The method of manufacturing the patient monitoring sensor of any one of examples 15-21, wherein disposing the light filter to cover the at least the portion of the active area of the detector comprises: at least one of coating or laminating an adhesive to a side of the light filter; and laminating the light filter to the active area of the detector via the adhesive.

Example 23. The method of manufacturing the patient monitoring sensor of example 22, wherein the adhesive comprises an optical index of refraction substantially matching an optical index of refraction of the plastic material.

Example 24. The method of manufacturing the patient monitoring sensor of any one of examples 15-23, wherein disposing the light filter to cover the at least the portion of the active area of the detector comprises: laminating the light filter to the sensor patch; and adhering the detector to the light filter.

Example 25. The method of manufacturing the patient monitoring sensor of any one of examples 15-24, wherein disposing the light filter to cover the at least the portion of the active area of the detector comprises encapsulating, by the light filter, the entire active area of the detector.

Example 26. The method of manufacturing the patient monitoring sensor of example 25, wherein encapsulating the entire active area of the detector comprises: integrating a light absorbing dye within an overmold material; and overmolding the material to form the light filter to encapsulate the entire active area of the detector.

Example 27. The method of manufacturing the patient monitoring sensor of any one of examples 15-26, wherein disposing the light filter to cover the at least the portion of the active area of the detector comprises: at least one of laminating or coating the light filter onto a wafer comprising a plurality of detector dies; dicing the plurality of detector dies; and encapsulating a detector die to form the detector.

Example 28. The method of manufacturing the patient monitoring sensor of any one of examples 15-27, wherein the light filter is configured to filter at least 80% of the second light wavelength, or at least 95% of the second light wavelength, or at least 99% of the second light wavelength.

Example 29. The method of manufacturing the patient monitoring sensor of any one of examples 15-26, wherein the light filter is configured to: pass at least 80% of the first light wavelength of the received first light from the tissue; and pass less than 5% of a cut-on light wavelength that is less than the threshold wavelength.

Example 30. The method of manufacturing the patient monitoring sensor of example 29, wherein the cut-on wavelength is 610 nm, wherein the threshold wavelength is 680 nm.

Example 31. A sensor comprising: a sensor patch configured to be applied to a region of skin of a patient, the sensor patch comprising: a flex circuit layer; and a bandage layer; a light source disposed on the flex circuit layer and configured to emit first light into a tissue of a patient, the first light comprising a first light wavelength greater than a threshold wavelength; a light filter configured to: receive the first light from the tissue of the patient; filter a second light wavelength from at least one of the received first light or ambient light, wherein the second light wavelength is less than or equal to the threshold wavelength; and output a filtered light that includes the received first light from the tissue; and a detector disposed on the flex circuit and configured to detect the filtered light, the filtered light being indicative of an oxygen saturation level of the patient.

Example 32. The sensor of example 31, wherein the light filter is configured to: pass at least 80% of the first light wavelength of the received first light from the tissue; and pass less than 5% of a cut-on light wavelength that is less than the threshold wavelength.

Example 33. The sensor of example 32, wherein the cut-on wavelength is 610 nm, wherein the threshold wavelength is 680 nm.

Example 34. The oximetry sensor of example 1, wherein the oximetry sensor comprises at least one of a pulse oximetry sensor or a regional oximetry sensor.

Example 35. The method of example 15, wherein the oximetry sensor comprises at least one of a pulse oximetry sensor or a regional oximetry sensor.

Example 36. The sensor of example 31, wherein the sensor comprises at least one of a pulse oximetry sensor or a regional oximetry sensor.

Various examples of the disclosure have been described. Any combination of the described systems, operations, or functions is contemplated. These and other examples are within the scope of the following claims.

What is claimed is:

1. An oximetry sensor comprising:
a light source configured to emit first light into a tissue of a patient, the first light comprising a first light wavelength greater than a threshold wavelength;
a light filter configured to:
receive the first light from the tissue of the patient;
filter a second light wavelength from at least one of the received first light or ambient light, wherein the second light wavelength is less than or equal to the threshold wavelength; and
output a filtered light that includes the received first light from the tissue; and
a detector configured to detect the filtered light, the filtered light being indicative of an oxygen saturation level of the patient.

2. The oximetry sensor of claim 1, wherein the filtered light comprises the first light wavelength and does not comprise the second light wavelength.

3. The oximetry sensor of claim 1, wherein the oximetry sensor has a form of a sensor patch configured to be applied to a region of skin of the patient.

4. The oximetry sensor of claim 3, wherein the sensor patch has a maximum dimension of less than 100 millimeters, or less than 75 millimeters, or less than 50, or less than 40 millimeters.

5. The oximetry sensor of claim 1, wherein the threshold wavelength is 600 nanometers.

6. The oximetry sensor of claim 1, wherein the light filter is configured to cover an entirety of an active area of the detector.

7. The oximetry sensor of claim 6, wherein the light filter is disposed on a surface of the active area of the detector, wherein the light filter comprises an alignment feature configured to register the light filter to cover the detector.

8. The oximetry sensor of claim 1, wherein the light filter comprises an absorption filter that comprises a light absorbing dye disposed on or within a plastic material.

9. The oximetry sensor of claim 1, further comprising a detector package with the detector, the light filter, and a potting material over the light filter.

10. The oximetry sensor of claim 1, wherein the light filter is configured to filter at least 80% of the second light wavelength, or at least 95% of the second light wavelength, or at least 99% of the second light wavelength.

11. The oximetry sensor of claim 1, wherein the light filter is configured to:
pass at least 80% of the first light wavelength of the received first light from the tissue; and
pass less than 5% of a cut-on light wavelength that is less than the threshold wavelength.

12. The oximetry sensor of claim 11, wherein the cut-on wavelength is 610 nm, wherein the threshold wavelength is 680 nm.

13. The oximetry sensor of claim 1, further comprising processing circuitry configured to generate information indicative of the oxygen saturation level based on the detected filtered light.

14. The oximetry sensor of claim 1, further comprising:
a flex circuit that supports the light source and the detector; and
a sensor assembly comprising a plurality of layers and the light filter laminated between adjacent layers of the plurality of layers, wherein the flex circuit is configured to couple to the sensor assembly to form the oximetry sensor.

15. A method of manufacturing an oximetry sensor, the method comprising:
attaching a light source to a sensor patch of the oximetry sensor, wherein the light source is configured to emit first light into a tissue of a patient, the first light comprising a first light wavelength greater than a threshold wavelength, wherein the sensor patch is configured to be applied to a region of skin of a patient;
disposing a light filter to cover over at least a portion of an active area of a detector, wherein the light filter is configured to:
receive the first light from the tissue of the patient;
filter a second light wavelength from at least one of the received first light or ambient light, wherein the second light wavelength is less than or equal to the threshold wavelength; and
output a filtered light that includes the received first light from the tissue; and
attaching the detector to the sensor patch, wherein the detector is configured to detect the filtered light, the filtered light being indicative of an oxygen saturation level of the patient.
